(19) United States
(12) Patent Application Publication
(10) Pub. No.: US 2020/0214964 A1
(43) Pub. Date: Jul. 9, 2020

(54) IMPROVEMENT IN OR RELATING TO ORGANIC COMPOUNDS

(71) Applicant: GIVAUDAN SA, Vernier (CH)

(72) Inventors: Amandine SCANDOLERA, Reims (FR); Romain REYNAUD, Toulouse (FR)

(21) Appl. No.: 16/638,007

(22) PCT Filed: Aug. 21, 2018
(86) PCT No.: PCT/EP2018/072514

§ 371 Date: Feb. 10, 2020

(30) Foreign Application Priority Data
Aug. 21, 2017 (GB) 1713362.0

(51) Int. Cl. A61K 8/73; A61Q 11/00; A61K 8/60

(52) U.S. Cl. CPC A61K 8/735; A61K 2800/92; A61K 8/602; A61Q 11/00

(57) ABSTRACT

The present invention relates to oral care compositions comprising hyaluronic acid, which are useful for promoting oral mucosa repair and/or for reducing biofilm formation.

### FIELD OF THE INVENTION

The present invention relates to oral care compositions comprising hyaluronic acid, which are useful for promoting oral mucosa repair and/or for reducing biofilm formation.

### BACKGROUND OF THE INVENTION

Oral care is the practice of keeping one's mouth clean and free of disease. This typically involves regular brushing, rinsing and cleaning of the oral cavity and in particular the teeth. It is important that oral hygiene be completed on a regular basis, as it can prevent dental disease from occurring. The most common types of dental disease are dental decay (also known as dental caries), gingivitis and periodontitis.

It is widely accepted that taking care of our teeth is essential to staying healthy. It is well known that lack of dental hygiene can lead progressively to tooth pain, tooth decay, tooth-loss and ultimately, without intervention, even more serious systemic health problems. In addition to the obvious physical health problems associated with poor dental hygiene, perhaps less appreciated is the relationship between the appearance of our teeth and our moods, as well as our social and mental well-being. Good teeth quality can have a huge impact on our mood because it inspires confidence and a willingness to smile more.

Nowadays, a large variety of oral care consumer products is available, including toothpastes, mouthwashes, chewing gums, oral sprays, and many more. Apart from cleaning, these products may also provide other effects, such as refreshing or whitening, for instance.

The oral tissue is frequently exposed to a number of sources of stress, such as mastication, speech, breathing, and bacterial invasion through oropharynx, nutrition, exterior environment, etc. These factors delay oral wound healing and increase the risk of infection.

Oral mucosa injuries are often related to tooth brushing, plaque removal, mastication, speech, ulcers, or surgery (e.g. for placing a dental implant or extraction of a tooth). Furthermore, excessive use of oral dental hygiene products, such as mouthwashes, may increase the disruption of oral tissue.

WO 2004/056346 discloses a method and compositions for wound management, wherein the latter comprise a chelating agent, a pH buffering agent, an antimicrobial agent, Vitamin E, a carrier and a surfactant. Among others, a mouth rinse for repairing a wound of the oral mucosa is disclosed.

EP 1 908 457 discloses compositions based on salt of hyaluronic acid for treating epithelial lesions.

Hyaluronic acid is an acid non-sulphurated mucopolysaccharide, being a basic constituent of the connective tissue. In man and animals, hyaluronic acid is present not only in the connective tissue, but also in important biological fluids, such as the vitreous humor, the aqueous humor and in the umbilical cord: it has not toxicity and no contraindications for use on man or on animals.

Hyaluronic acid can be obtained by extraction from natural substances, for example from cocks' crests, or can be produced by biotechnology methods. It has a wide molecular weight spectrum which can reach 15′000 kDa, depending on the method for its production. Hyaluronic acid is known to be used as the sodium or potassium salt in human therapy and in cosmetics: exogenous application of hyaluronic acid has a beneficial effect favouring connective organization and is also effective in reducing or eliminating inflammatory processes induced by germs producing hyaluronase, it facilitates resolution of phlogistic components, reduces excessive capillary permeability, accelerates tissue repair processes and develops an antiedematogenic action by metabolically binding free water to its molecular structures.

In the cosmetic field, hyaluronic acid is used for its invigorating, tonic, skin repair and hydrating properties.

EP 0 138 572 discloses the use of hyaluronic acid fractions having an average molecular weight from about 50 to about 100 kDa for use in the healing of tissue wounds.

EP 0 444 492 illustrates the use of hyaluronic acid of high molecular weight (between 88 and 4′000 kDa) for the treatment and prevention of inflammatory states of the oral mucosa.

Healthy oral flora is typically composed of more than 700 bacterial species. The distribution of bacteria depends on the surface of the oral cavity: periodontal, gingival, dental plaque, palate, saliva, etc. Usually, Streptococcus spp. is the most predominant bacteria. In certain areas, mainly in fissures of teeth, supra and subgingival plaque, Staphylococcus spp. was found regularly, but Staphylococcus spp. is only a transient resident of the oral microflora. Among others, Staphylococcus aureus is found in the oral cavity. This is a transient but frequent bacterial resident of oral cavity, with an incidence of about 24 to 84% in healthy adult dentate oral cavities and of about 48% among the denture-wearing population.

Oral infections presumably occur through cross-infection from a variety of sources. They may cause pathologies, such as angular cheilitis, parotitis, or staphylococcal mucositis. In these areas, Staphylococcus aureus was found.

The aim of the present invention is to provide an improved oral care composition that protects the oral tissue and furthers wound healing.

### SUMMARY OF THE INVENTION

In a first aspect, the present invention provides an oral care composition comprising hyaluronic acid having an average molecular weight of less than 500 kDa.

Surprisingly, it was found that hyaluronic acid of low and intermediate molecular weight is very effective in oral tissue repair and protection against biofilm.

In a second aspect, the present invention provides a method of reducing biofilm formation on an oral surface by applying an oral care composition according to the present invention to the oral surface.

### DETAILED DESCRIPTION OF THE INVENTION

The present invention provides an oral care composition comprising hyaluronic acid, wherein the hyaluronic acid has an average molecular weight of less than 500 kDa.

Throughout the present disclosure, the term “average molecular weight” is meant to refer to the weight average molecular weight, unless noted otherwise.

The term “oral care composition”, as used herein, refers to non-food compositions that are designed to be taken into the mouth to deliver a variety of benefits. “Oral care composition” does not only ready-to-use consumer products include, but also precursors of consumer products (e.g. stock solutions that need to be diluted prior to use) and active parts of such consumer products. Essentially, any composition suitable for and useful in the treatment of the oral cavity is meant to be covered by this term.

Such compositions include dentifrices, mouthwashes, mouth sprays and gargle compositions, breath strips (edible films placed in the oral cavity to administer thereto an active agent such as a flavourant or breath-freshening agent), and chewing gums.

The term “dentifrice”, as used herein, means toothpaste, oral care gels or liquids, unless otherwise specified. The dentifrice composition may be a single-phase composition or it may be a combination of two or more separate dentifrice compositions. The dentifrice composition may be in any desired form, such as deep striped, surface striped, multi-layered, having the gel surrounding the paste, or any combination thereof.

Oral care products include, for example, toothpaste, mouthrinse, mouthwash, and portable “on the go” oral malodour control products including chewing gum, candies, pastilles, edible films, and oral sprays. Formulations for the above-mentioned oral care products are well-known in the art. Oral care products contain excipients including, for example, surfactants, emulsifiers, solvents, colorants, preservatives, antioxidants, antimicrobial agents, enzymes, vegetal or mineral oils, fats, proteins, solubilisers, sugar derivatives, vitamins, polyols including sorbitol, organic acids, artificial sweeteners, polymers, thickeners, chewing gum gum bases, oral care actives including fluorine compounds, and zinc salts (for example zinc gluconate, zinc acetate, zinc citrate). Some oral care products contain alcohols, in particular lower alcohols (C1-C4).

It was found that hyaluronic acid of both low and intermediate molecular weight is able to promote the oral mucosa repair after 24 h of treatment in comparison with untreated mucosa. The effects involved an active mechanism of wound healing coupled to a full recovery of the injury. A detailed description of the experiments preformed to assess re-epithelization after injury can be found in examples 1-4 below.

In contrast thereto, hyaluronic acid of high molecular weight (1′000 to 1′400 kDa) only demonstrated a mechanic effect on tissue repair by forming a film on the skin surface.

Consequently, the oral care composition of the present invention comprises hyaluronic acid having an average molecular weight of less than 500 kDa, more preferably of less than 400 kDa, and even more preferably of less than 300 kDa.

The oral care composition of the present invention was also found to provide enhanced mucosal tactile properties. In particular, the smoothness of the gums was improved and the composition provided a “healthy feel”.

In comparative testing (see example 11 below), the oral care compositions of the present invention were perceived to provide a more pleasant, cleaner, and less drying feel, and to feel like they were taking care of one's mouth and gums. It was also found that the incorporation of hyaluronic acid had advantages effects on the taste of toothpastes, making them less bitter, sweeter and less salty. They were also experienced as less foaming, less burning and less drying, and provided a cleaner feel.

It was further found that the oral care compositions of the present invention exhibit reduced astringency, in particular those containing zinc salts. Zinc salts are commonly used in oral care formulations, and are in many cases considered to be an issue with oral care consumers due to a related astringency. It has now been found that, by incorporating the hyaluronic acid as defined herein, the astringent and/or drying effects of toothpastes, mouthwashes, and other oral care products can be reduced or even completely eliminated.

Preferably, the oral care composition of the present invention comprises hyaluronic acid having an average molecular weight of at least about 5 kDa.

Preferred ranges for the average molecular weight are about 20 to about 50 kDa for low molecular weight hyaluronic acid and about 100 to about 300 kDa for intermediate molecular weight hyaluronic acid.

Low molecular weight hyaluronic acid was found to be particularly effective for promoting oral mucosa repair.

Consequently, in a preferred embodiment, the oral care composition of the present invention comprises hyaluronic acid having an average molecular weight of less than about 80 kDa, preferably of about 10 to about 65 kDa, and most preferably of about 20 to about 50 kDa.

The biological activity of low molecular weight hyaluronic acid is related to cell cohesion (increasing the expression of Tigh junction (ZO-1/Occludine)), plumping (boosting the production of type I collagen), hydration (increasing the water content of the skin), mechanical properties (improvement of firmness and tonicity), and skin penetration (significant skin penetration (120 μM)).

It was further found that intermediate molecular weight hyaluronic acid also decreased the bacterial adhesion and their penetration into the tissue from a colonized oral mucosa model. The biofilm analysis by scanning electronic microscopy showed that the oral mucosa treated with intermediate molecular weight hyaluronic acid presented fewer bacterial clusters and blocked the switch from planktonic phenotype to biofilm observed by the matrix polysaccharide production. Thus, intermediate molecular weight hyaluronic acid not only promoted the tissue repair of oral mucosa, but also prevented the biofilm formation from pathogenic bacteria.

Consequently, in a preferred embodiment, the oral care composition of the present invention comprises hyaluronic acid having an average molecular weight of about 80 to about 500 kDa, preferably of about 90 to about 400 kDa, and most preferably of about 100 to about 300 kDa.

The biological activity of intermediate molecular weight hyaluronic acid is related to antimicrobial defence (increasing of Defensine production depending on TRL2 and TRL4 in vitro and ex vivo; controlling the cutaneous immunity by bacterial inhibition of E. Coli), cell proliferation and migration (boosting the cell proliferation and migration (keratinocytes and fibroblasts)), and skin penetration (significant penetration into the skin (40 μM)).

Detailed results of the performed experiments are described in examples 5-9 below. In conclusion, it was found that intermediate molecular weight hyaluronic acid
Restores the TEER level without colonization (barrier function)
Decreases the total bacterial count (−50%; bacterial proliferation)
Limits the bacterial invasion in RHO (bacterial penetration)
Conserves the planktonic phenotype in bacteria (biofilm formation)
Inhibits the biofilm formation (polysaccharidic matrix; biofilm formation)

Overall, it was found that intermediate molecular weight hyaluronic acid was the best for oral care application because it was able to actively boost the oral tissue repair and showed an efficient protection against bacterial biofilm. Therefore, according to a particularly preferred embodiment, the oral care composition of the present invention comprises intermediate molecular weight hyaluronic acid, and in particular comprises hyaluronic acid having an average molecular weight of about 100 to about 300 kDa.

In the oral care composition of the present invention, the hyaluronic acid may be provided in any suitable form known to the person skilled in the art. In particular, it may be provided neat, as a solution or suspension, in encapsulated or mycellized form, adsorbed on particulate surfaces, or otherwise distributed. If the hyaluronic acid is used in neat form, then it is typically a powder, which may be directly incorporated into the oral care composition. Alternatively, the hyaluronic acid may be used in the form of a solution or suspension, preferably an aqueous or alcoholic solution or suspension. The hyaluronic acid may also be provided in encapsulated or mycellized form, preferably in the form of a slurry. Alternatively, the hyaluronic acid may be adsorbed on particulate surfaces, e.g. on titanium dioxide, or otherwise distributed.

The oral care composition of the present invention may further contain other active ingredients, as well as additives generally known to the person skilled in the art. In particular, it may further comprise disinfectants, astringents, haemostatics, oral malodour counteractants, and mixtures thereof.

The oral care compositions of the present invention may also contain intense flavours to mask oral malodour, or rather its perception, by using a dominating flavour or odour, while the malodour remains present but is less detectable in combination. For example, JP 2004018431 describes various flavour compositions comprising mint oils or compounds known to be comprised in mint plants, which are known actives against halitosis (for example menthol), in combination with masking flavour compounds.

The oral care compositions of the present invention may also contain classical antibacterial agents, such as Triclosan, cetyl-pyridinium chloride, and chlorhexidine. Also, the antibacterial effect of natural ingredients or flavour compounds may be used. These include, for example, thymol, wintergreen oil, methyl salicylate, eucalyptol and mint oils and compounds occurring in mint plants, in particular menthol. Further natural ingredients that are known to have a malodour counteracting effect include parsley, combinations of ionones (alpha-ionone, beta-ionone, gamma-ionone, dihydroionone, alpha-methylionone, irone) with zinc salts, certain higher alcohols (in particular nonanol) in combination with C1-04 alcohols (WO 99/51093).

An alternative is to reduce oral malodour by means that leave the oral bacteria largely intact, in particular by chemically capturing the malodorous volatiles by reactive chemicals. For example, polyphenolic compounds such as those contained in green tea extract or zinc salts may be used to capture volatile sulphur compounds. A further chemical approach is to degrade the malodorous sulphur volatiles by applying oxidizing agents.

Another approach is by enzymatic inhibition of the relevant bacterial enzyme(s) so that the malodorous sulphur volatiles are not formed in the first place. For example, certain plant extracts (tomato, Uncaria gambier, Quillaja saponaria, Hamamelis virginiana, Eriobotrya japonica, Equisetum arvense, Crataegus oxyacantha, Diospyros kaki, Curcuma domestica, Ginkgo biloba, green tea, black tea, and/or oolong tea) can be used to inhibit the methioninase enzyme which generates MeSH.

The oral care compositions of the present invention may also comprise oral malodour counteracting actives selected from the group consisting of oct-2-ynoic acid methyl ester, non-2-ynoic acid methyl ester, oct-2-enoic acid ethyl ester, oct-2-enoic acid methyl ester, non-2-enoic acid methyl ester, hex-2-enoic acid ethyl ester, hex-2-enoic acid methyl ester, non-2-ynoic acid ethyl ester, non-2-enoic acid ethyl ester, hept-2-enoic acid ethyl ester, and hept-2-enoic acid methyl ester.

The oral care composition of the present invention may further comprise one or more actives selected from the group consisting of ionone, alpha ionone, beta ionone, zinc salts, polyphenolic compounds, and antibacterial agents.

Antibacterial agents may be selected from the group consisting of triclosan, cetylpyridinium chloride, polyhexidine bisguanide, chlorhexidine, and antibacterial flavour materials. Antibacterial flavour materials include in particular thymol, carvacrol, eugenol, isoeugenol, cinnamic aldehyde, menthol. Flavour materials may be provided in form of an essential oil containing these ingredients. Preferred essential oils include oil from thyme, origanum, clove, cinnamon leave, cinnamon bark, parsley seed, parsley leaf, mint, spearmint, and peppermint.

Useful polyphenolic compounds are, for example, those that comprise a gallate moiety, in particular epigallocatechin gallate. These may be in form of certain natural ingredients, in particular in green tea and its extract, for example green tea extract enriched in epigallocatechin gallate. In particular, an OMC flavour in particulate form may be formed by spray-drying an OMC flavour composition and mixing it with green tea particles to form a dry blend of green tea and OMC flavour composition. The resulting particulate material can be easily admixed to an OMC product formulation.

The oral care composition of the present invention may further comprise one or more actives selected from the group consisting of 5-isopropyl-2-methyl-phenol, octan-1-ol, 3,7-dimethyl-oct-6-en-1-ol, 3,7-dimethyl-octan-1-ol, 1-isopropyl-4-methyl-cyclohex-3-enol, 3,7-dimethyl-octa-2,6-dien-1-ol, 2-(4-methyl-cyclohex-3-enyl) propan-2-ol, 3,7-dimethyl-octa-1,6-dien-3-ol, nona-2,4-dienal, non-2-enal, 2,6,6-trimethyl-cyclohex-1-enecarbaldehyde, 3-(4-isopropyl-phenyl)-2-methyl-propionaldehyde, 4-isopropenyl-cyclohex-1-enecarbaldehyde, 5-methyl-2-phenyl-hex-2-enal, 4-methoxy-benzaldehyde, 2,6-dimethyl-hept-5-enal, dec-2-enal, phenyl-acetaldehyde, 2-phenyl-propionaldehyde, 3,7,11-trimethyl-dodeca-1,3,6,10-tetraene, 3,7-dimethyl-octa-1,3,6-triene, 1-isopropyl-4-methyl-cyclohexa-1,3-diene, 1-methyl-4-(5-methyl-1-methylene-hex-4-enyl)-cyclohexene, 1-isopropyl-4-methylbenzene, dec-3-en-2-one, 3-methyl-2-pentyl-cyclopent-2-enone, 6-methyl-hepta-3,5-dien-2-one, acetic acid octyl ester, acetic acid oct-2-enyl ester, 2-methyl-but-2-enoic acid hex-3-enyl ester, acetic acid nonyl ester, acetic acid heptyl ester, butyric acid 3-phenyl-allyl ester, acetic acid 1,7,7-trimethyl-bicyclo[2.2.1]hept-2-yl ester, acetic acid 4-allyl-2-methoxy-phenyl ester, acetic acid 1-methyl-1-(4-methyl-cyclohex-3-enyl)-ethyl ester, acetic acid 2-isopropenyl-5-methyl-cyclohexyl ester, 5-octyl-dihydro-furan-2-one, 1,1-dimethoxy-3,7-dimethyl-octa-2,6-diene, 1-allyl-4-methoxy-benzene, 6-hexyl-tetrahydro-pyran-2-one, 3-butyl-3H-isobenzofuran-1-one, 2-pentyl-furan, (2E, 5E/Z)-5,6,7-trimethylocta-2,5-dien-4-one, 4-methyl-dec-3-en-5-ol, 1-cyclopropylmethyl-4-methoxy-benzene, origanum essential oil, galbanum essential oil, litsea cubeba essential oil, tagete essential oil, jasmin absolute, lavande essential oil, lavandin essential oil, rosemary essential oil, and vetiver essential oil.

The combination of enzyme-inhibiting oral malodour counteracting substances with certain antibacterial flavours has been found to be particularly beneficial and results in compositions both highly effective and pleasantly tasting. Accordingly, The oral care composition of the present invention may further comprise up to 50%, or up to 90% (w/w) of total flavour ingredients, one or more flavours with antibacterial properties selected from the group consisting of menthol, thymol, eugenol, 5-isopropyl-2-methyl-phenol, octan-1-ol, 3,7-dimethyl-oct-6-en-1-ol, 3,7-dimethyl-octan-1-ol, 1-isopropyl-4-methyl-cyclohex-3-enol, 3,7-dimethyl-octa-2,6-dien-1-ol, 2-(4-methyl-cyclohex-3-enyl) propan-2-ol, and 3,7-dimethyl-octa-1,6-dien-3-ol. The compounds can be added in the form of pure compounds or in the form of natural ingredients (for example essential oils from plants, such as mint oils, peppermint oil, or spearmint oil for menthol or thyme oil for thymol).

As an example, a toothpaste composition may further comprise other compounds commonly used in toothpaste, such as oral disinfectant, abrasive, humectant, detergent, binder, frothing agent, sweetening agent, preservative, buffering agent, flavours and cooling agents and may be prepared following the procedures known to the skilled person.

The oral care compositions of the present invention may also comprise one or more additional ingredients or excipients conventionally used in conjunction with oral care compositions, for example flavour compounds, excipients, solvents, cooling agents for providing a fresh mouthfeel and/or other auxiliary agents commonly used in the art.

Examples of known flavour ingredients may be found in one of the FEMA (Flavour and Extracts Manufacturers Association of the United States) publications or a compilation thereof which is available from and published by FEMA and contains all FEMA GRAS (Generally Regarded As Safe) publications from 1965 to present, e.g. GRAS 21 published 2003, or in Allured's Flavor and Fragrance Materials 2004, published by Allured Publishing Inc.

Examples of known excipients for oral care products may be found in Gaffar, Abdul, Advanced Technology, Corporate Technology, Department of Oral Care, Colgate-Palmolive Company, Piscataway, N.J., USA. Editor(s): Barel, Andre O.; Paye, Marc; Maibach, Howard I., Handbook of Cosmetic Science and Technology (2001), p. 619-643. Publisher: Marcel Dekker, Inc., New York, N. Y, and in Cosmetics: Science and technology, 2nd edition, p. 423-563. Edited by M. S. Balsam and E. Sagarin, Wiley Interscience, 1972.

Particular examples of cooling agents may include, but are not limited to, menthol, menthone, isopulegol, N-ethyl p-menthanecarboxamide (WS-3), N,2,3-trimethyl-2-isopropylbutanamide (WS-23), menthyl lactate, menthone glycerine acetal (Frescolat® MGA), mono-menthyl succinate (Physcool®), mono-menthyl glutarate, 0-menthyl glycerine (CoolAct® 10), 2-sec-butylcyclohexanone (Freskomenthe®) and 2-isopropyl-5-methyl-cyclohexanecarboxylic acid (2-pyridin-2-yl-ethyl)-amide. Further examples of cooling agents can be found e.g. in WO 2006/125334 and WO 2005/049553, which are incorporated by reference.

In a preferred embodiment, the oral care composition of the present invention further comprises at least one thymol glycoside, and in particular thymol α-glucoside. The corresponding British patent application filed on the same day is herewith incorporated by reference. It was found that thymol glycosides provide a long lasting antiseptic effect and improved liking in comparison to thymol. Furthermore, their water solubility is significantly better than that of thymol.

The oral care composition of the present invention should comprise the hyaluronic acid in a concentration to be effective. In particular, the oral care composition may comprise the hyaluronic acid in a concentration of at least 0.1% (w/v), more preferably in a concentration of at least 0.2% (w/v). In a preferred embodiment, the oral care composition comprises the hyaluronic acid in a concentration of 0.1 to 1.0% (w/v), more preferably in a concentration of 0.2 to 0.5% (w/v). Higher concentrations are also possible, but lead to an increase in costs. It is particularly preferred that the low molecular weight hyaluronic acid is used in a concentration of 0.5% (w/v) and that the intermediate molecular weight hyaluronic acid is used in a concentration of 0.2% (w/v).

Throughout this disclosure, concentrations are indicated as percentages weight per volume (% w/v), unless stated otherwise.

In a further aspect, the present invention also provides an oral care composition for promoting oral mucosa repair. Said composition preferably comprising hyaluronic acid having an average molecular weight of less than 500 kDa.

More preferably, the oral care composition for promoting oral mucosa repair comprises hyaluronic acid having an average molecular weight of about 10 to about 400 kDa, more preferably of about 20 to about 300 kDa. In particular, the oral care composition for promoting oral mucosa repair comprises hyaluronic acid having an average molecular weight of about 20 to about 50 kDa (low molecular weight hyaluronic acid) or of about 100 to about 300 kDa (intermediate molecular weight hyaluronic acid). Low molecular weight hyaluronic acid is particularly preferred.

Thus, the present invention also refers to the use of hyaluronic acid having an average molecular weight of less than 500 kDa for the manufacture of an oral care composition for promoting mucosa repair.

In a further aspect, the present invention also provides a method of promoting oral mucosa repair, in particular a non-therapeutic one. Said method involves the application of an oral care composition according to the present invention to the oral mucosa.

In a further aspect, the present invention also provides an oral care composition for reducing biofilm formation. Said composition preferably comprises hyaluronic acid having an average molecular weight of less than 500 kDa.

More preferably, the oral care composition for reducing biofilm formation comprises hyaluronic acid having an average molecular weight of about 10 to about 400 kDa, more preferably of about 20 to about 300 kDa. In particular, the oral care composition for promoting oral mucosa repair comprises hyaluronic acid having an average molecular weight of about 20 to about 50 kDa (low molecular weight hyaluronic acid) or of about 100 to about 300 kDa (intermediate molecular weight hyaluronic acid). Intermediate molecular weight hyaluronic acid is particularly preferred.

Thus, the present invention also refers to the use of hyaluronic acid having an average molecular weight of less than 500 kDa for the manufacture of an oral care composition for reducing biofilm formation.

In a further aspect, the present invention also provides a method of reducing biofilm formation on an oral surface, in particular a non-therapeutic one. Said method involves the application of an oral care composition according to the present invention to the oral surface.

The present invention is further illustrated by means of the following non-limiting examples:

### Example 1: Sample Preparation and Treatment with Hyaluronic Acid

Experiments were performed on Reconstituted Human Oral Epithelium (RHO).

Immediately after the arrival in the laboratory, the RHO was removed from the agarose nutrient solution under a sterile airflow cabin. The inserts were rapidly placed in a 6-well plate previously filled with 1 ml of the maintenance medium at room temperature. The wells were placed in an incubator at 37° C., 5% CO₂ and saturated humidity overnight.

On the next day, an injury was performed with a glass capillary.

The RHO samples were then treated with a solution containing hyaluronic acid having low (20-50 kDa), intermediate (100-300 kDa) or high (1′000-1′400 kDa) molecular weight. Untreated RHO was used as a control without treatment.

### Example 2: Assessment of Re-Epithelization after Injury by TEER (Trans Epithelial Electrical Resistance)

The RHO samples for this assessment were prepared according to the procedure of example 1.

24 h after the treatment with hyaluronic acid, the trans epithelial electric resistance (TEER) was measured as follows:

0.5 ml of physiological saline solution was directly applied on the tissue placed in a 6-well plate containing 5 ml of saline solution as well. The two electrodes of a Millicell-ERS Voltohmmeter (range 0-20 kΩ) were placed were placed in two compartments on either side of the tissue, such that the electric flux passed through the tissue. The result of the measurement directly appeared on the display.

Three measurements for each tissue were performed because of the variability within the tissues. The blank value (insert without tissue) was subtracted from the sample values (mean from 3 measurements). This result was then corrected considering the size of the tissue surface (0.5 cm²):

Ωₛₐₘₚₗₑ−Ω_{blank}=Ω*0.5 cm²

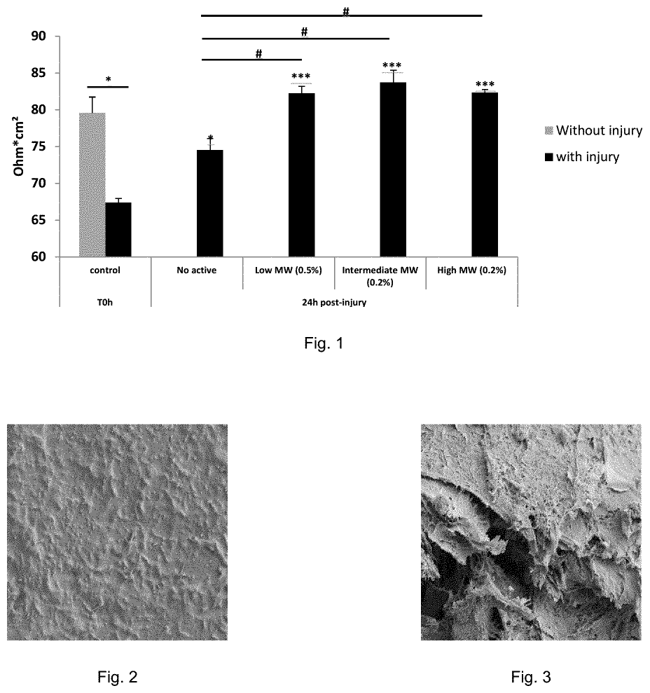

The results are shown in FIG. 1.

As can bee seen from FIG. 1, the TEER values measured for the samples treated with hyaluronic acid were significantly higher than for the control sample that was not treated after injury.

TEER reflects the skin barrier function.

In conclusion, it was found that low and intermediate molecular weight hyaluronic acid improved the barrier function after an injury, up to the point of reaching the value before injury. Without being bound by theory, it is believed that the improvement of barrier function is due to an increase of tissue repair caused by the hyaluronic acid.

### Example 3: Assessment of Re-Epithelization after Injury by Scanning Electronic Microscopy (×10000)

The RHO samples for this assessment were prepared according to the procedure of example 1.

24 h after treatment with hyaluronic acid, the samples for scanning electronic microscopy (SEM) were immediately fixed by immersion in 2.5% glutaraldehyde in phosphate buffered saline (PBS) for 24 h, washing in 0.1 M sodium cacodylate buffer, pH 7.4, and then carried out in 1% osmium tetraoxide (OSO₄) in the same buffer (2 h at room temperature). The samples were dehydrated in ascending grades of ethanol at room temperature and Hexamethyldisilazane overnight.

The samples were placed on pins with carbon tabs, coated with a layer of gold using a Polaron Equipment limited SEM coating unit E5100, and then transferred to a SEM Zeiss Sigma Electron Microscope for viewing and photography. Magnification of 10000× was performed.

Results are shown in the following figures:

FIG. 2: control without injury, at time T0 h;

FIG. 3 control with injury, at time T24 h;

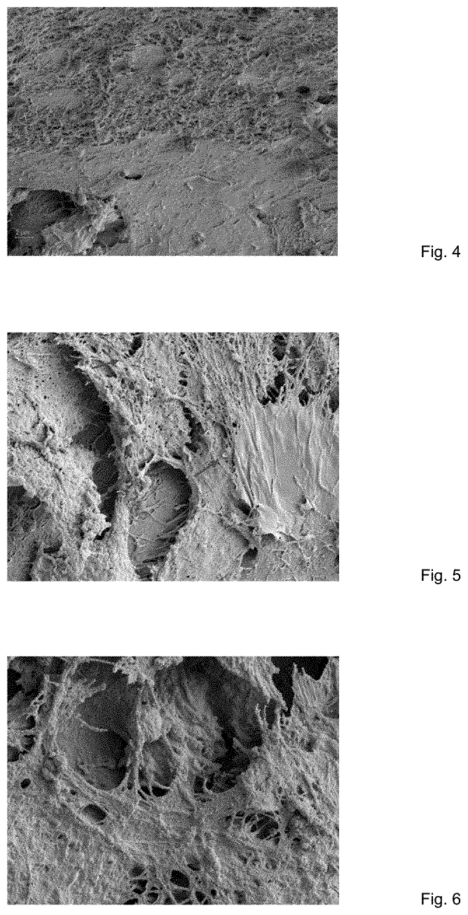

FIG. 4: after injury and treatment with low molecular weight hyaluronic acid (20-50 kDa; 0.5%), at time T24 h;

FIG. 5 after injury and treatment with intermediate molecular weight hyaluronic acid (100-300 kDa; 0.2%), at time T24 h; and

FIG. 6: after injury and treatment with high molecular weight hyaluronic acid (1′000-1′400 kDa; 0.2%), at time T24 h.

As can be seen from FIG. 4, low molecular weight hyaluronic acid led to a recovery of the wound. Many new extracellular matrix (ECM) fibers almost completely covered the wound. Microvillis were present (blue arrow), indicating homeostasis recovery in terms of tissue moisturization. Overall, a good progress of tissue repair was observed.

As can be seen from FIG. 5, intermediate molecular weight hyaluronic acid caused flattened cells to migrate near the wound. These cells participate in the repopulation and formation of a bridge of matrix fibers. Overall, the pictures showed an active process of tissue repair.

In contrast thereto, as can be seen from FIG. 6, the repair process was significantly less advanced for high molecular weight hyaluronic acid. Instead, film forming was observed.

### Example 4: Assessment of Re-Epithelization after Injury by Immunohistochemistry on ZO-1 and Integrin B1

The RHO samples for this assessment were prepared according to the procedure of example 1.

At the end of the exposures (at least overnight), the tissue samples were fixed in buffered 10% formalin. Samples were included in paraffin blocks and sections of 5 μm were prepared. These slides were stained with Haematoxilin and Eosin.

The used technique allows for visualization of a specific protein or antigen in cells or tissue sections by binding a specific antibody chemically conjugated with a fluorescent dye. The indirect immunofluorescence staining is a procedure, in which a secondary antibody labelled with a fluorochrome is used to recognize a primary antibody. Immunofluorescence staining can be performed on cells fixed on slides and tissue sections. Immunofluorescence stained samples are examined under a fluorescence microscope or confocal microscope. The specificity of both the immuno-localisations is demonstrated in the slides where the primary and secondary antibodies are replaced by saline solution.

The following antibodies were used:

ZO-1: Rabbit polyclonal antibody anti-ZO-1 (Invitrogen, 61-7300), diluted at 5 μg/ml for an overnight incubation at 4° C. in 1% bovine serum albumin (BSA) in PBS; and the secondary antibody in Alexa Fluor 555 donkey anti-rabbit (Invitrogen, A31572). The nuclei were stained with (4′,6-diamidino-2-phenylindole) (Dapi).

INTEGRIN (ITGB1): Mouse monoclonal antibody anti-Integrin beta 1 (Abcam, ab3167) diluted at 2 μg/ml for a 2 h incubation at room temperature in 1% BSA in PBS; and the secondary antibody in Alexa Fluor 488 goat anti-mouse (Invitrogen, A10680). The nuclei were stained with Dapi.

The slices were examined under a Leica DM 2500 FLUO microscope and analyzed by Leica LAS software.

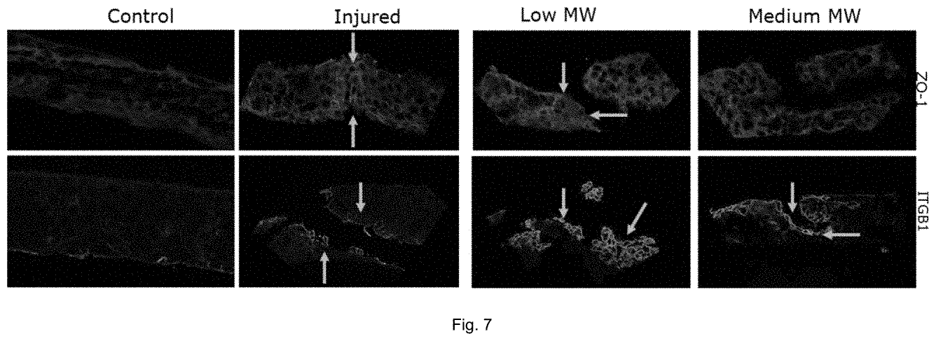

The results are shown in FIG. 7.

The Control from example 1 was used as no injury tissue control.

“Injured” refers to a sample where normal wound healing took place without any treatment. It was found that injury increased the ITGB1 and ZO-1 expression, corresponding to a normal wound healing process.

An incubation with low molecular weight hyaluronic acid induced an over-expression of ZO-1 and Integrin beta 1, two key actors in the wound healing process. These results suggest that the tissue repair process is boosted in comparison with the injured control.

The effects obtained with hyaluronic acid of intermediate molecular weight showed that this boosts the wound healing process by over-expression of integrin beta 1 exclusively. These effects are less pronounced than those with low molecular weight hyaluronic acid.

Thus, is was found that low and intermediate molecular weight hyaluronic acid induced an active mechanism of wound healing, involving the over-expression of Integrin B1 and ZO-1.

### Example 5: Culturing of Reconstituted Human Oral Epithelium (RHO) Samples

Immediately after the arrival in the laboratory, the RHO was removed from the agarose nutrient solution under a sterile airflow cabin. The inserts were rapidly placed in a 6-well plate previously filled with 1 ml of the maintenance medium containing antibiotics at room temperature. The wells were placed in an incubator at 37° C., 5% CO₂ and saturated humidity overnight.

The protocol was performed on duplicate tissues for bacterial burden and on single tissue for further morphological analysis (SEM, H&E).

S. aureus MRSA ATCC 33591 was thawed and cultured in nutrient broth at 37° C., under agitation.

30 μL of intermediate molecular weight hyaluronic acid solution (0.2% w/v) or of chlorhexidine (CHL) 0.2% (positive control), respectively, were topically applied to RHO samples, and incubated for 24 h. Then, the product residual volume was removed by a micropipette from the epithelium surface.

The RHO samples were then colonized with S. Aureus bacterial suspensions (O.D. 0.1 about 10⁶ UFC/tissue), which was applied topically for 4 h. After 4 h, the remaining S. aureus solution was removed and RHO tissue samples were incubated for 16 h at 37° C., 5% CO₂.

### Example 6: Assessment of Biofilm Formation by TEER (Trans Epithelial Electrical Resistance)

The samples for this assessment were prepared according to the procedure of example 5.

TEER was measured as described hereafter: 0.5 ml of physiological saline solution was directly applied on the tissue placed in a 6-well plate containing 5 ml of saline solution as well.

The two electrodes of a Millicell-ERS Voltohmmeter (range 0-20 kΩ) were placed in two compartments on either side of the tissue, such that the electric flux passed through the tissue.

Three measurements for each tissue sample were conducted because of the variability within the tissues.

The blank value (insert without tissue) was subtracted from the sample value (mean from 3 measurements). This result was then corrected considering the size of the tissue surface (0.5 cm²):

Ωₛₐₘₚₗₑ−Ω_{blank}=Ω*0.5 cm²

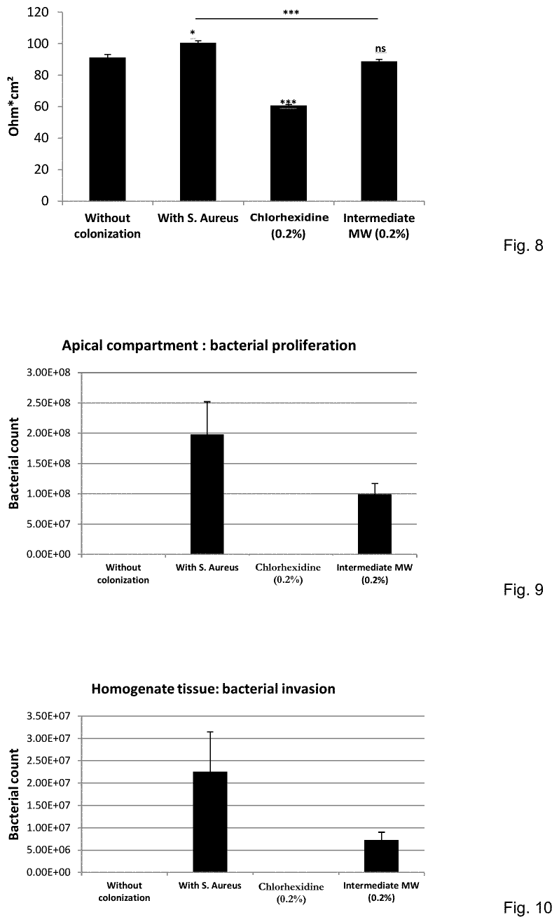

The results are shown in FIG. 8:

Colonization by S. aureus induced slight increasing of TEER. Without being bound by theory, it is assumed that the RHO thickness is increased due to the colonization.

For the sampled treated with CHL, TEER decreased, what means that the barrier function was altered (possibly due to toxicity).

The addition of intermediate molecular weight hyaluronic acid did not affect TEER; the result was similar to the control.

In conclusion, the intermediate molecular weight hyaluronic acid limited the bacterial growth and did not affect the barrier function.

### Example 7: Assessment of Biofilm Formation by Bacterial Count

The samples for this assessment were prepared according to the procedure of example 5. In order to perform the bacterial count on apical, basolateral and homogenate compartments, the following procedure was used:
For the basolateral compartment: 1 ml of culture medium from each well was sampled.
For the apical compartment, the samples from the TEER measurement according to example 6 were used: Each sample contained 500 μl of physiological saline solution. These samples were moved to a new 6-well plate that had been previously filled with 2 ml/well of saline solution and put in a sonicator bath for 7 minutes at 40 kHz. After sonication, 500 μl of saline solution were sampled for the apical compartment and the RHO tissues were rinsed twice with 200 μl of saline solution. The total amount of solution (900 μl) was then pooled together to obtain the harvested apical compartment.
For the homogenate compartment: a sterile scalpel blade has been used to harvest tissues from the insert and to place them in an Eppendorf tube containing 500 μL of 0.5% Triton X-100 solution prepared in sterile distilled water for at least 10 minutes.

Bacterial counts of the three compartments were performed on Nutrient Agar plates, plating the appropriate 10-fold dilutions (from the not diluted to 10-7) of each compartment suspension (one 10 μl drop for each dilution). After 1 day of incubation at 37° C., the colonies were numbered visually.

The results of the bacterial count are shown in FIG. 9 (apical compartment) and 10 (homogenate tissue).

Colonization by S. Aureus induced an increase of bacterial count in both apical and homogenate compartment.

Treatment with CHL showed a strong bactericidal property.

Treatment with hyaluronic acid led to a decrease of the bacterial count by about 50% in both the apical and homogenate compartment. Thus, it had an impact on the bacterial proliferation and limited the bacterial adhesion and penetration.

In conclusion, hyaluronic acid was able to generate a good protection against bacterial penetration.

### Example 9: Biofilm Visualization by Scanning Electronic Microscopy

The samples for this experiment were prepared according to the procedure of example 5.

After treatment, samples for SEM were immediately fixed by immersion in 2.5% gluteraldehyde in PBS. Slides were washed three times in 0.065 M phosphate buffer, then placed in 1% OSO₄ in phosphate buffer at 0.064 M (pH 7.4). The samples were dehydrated through a graded series of ethanol, and then critical-point dried in a CO₂ liquid Bemar SPC 1500 apparatus. The samples were mounted on a stub, hand painted with gold, and observed with a Cambridge Mark 250 SEM. Magnification of 10000× was performed.

Results are shown in the following figures:

FIG. 11: control without colonization;

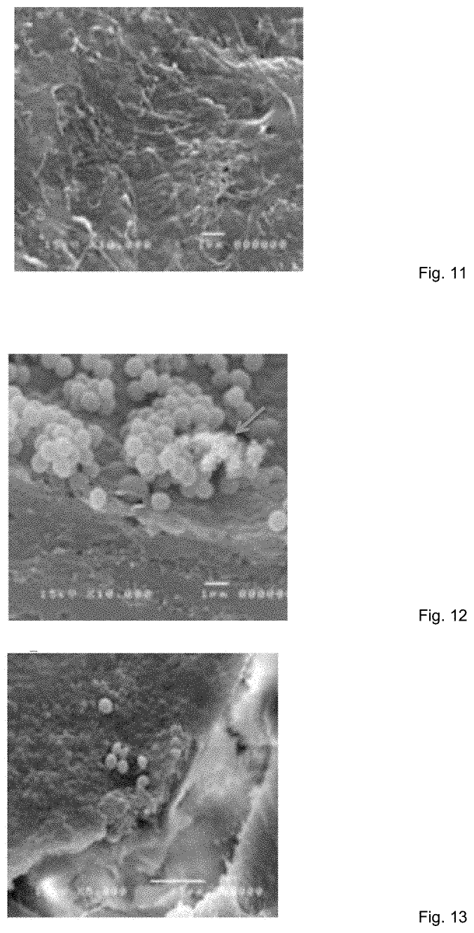

FIG. 12: colonization with S. Aureus after 4 h; and

FIG. 13: colonization with S. Aureus and treatment with intermediate molecular weight hyaluronic acid (100-300 kDa; 0.2% w/v), at time after 4 h.

FIG. 11 shows Reconstituted Human Oral Epithelium (RHO) without bacterial colonization.

FIG. 12 shows colonies in cluster and a planktonic morphology. The bacteria start to produce polysaccharide matrix to shift from planktonic to biofilm phenotype.

FIG. 13 shows that less colonies are in cluster and a planktonic morphology. The bacteria counteract the biofilm formation. No polysaccharide matrix is visible. Thus, the planktonic phenotype is conserved.

### Example 10: Deposition of Hyaluronic Acid in Oral Mucosa

Mouthwashes with and without hyaluronic acid were tested on three volunteers according to the following protocol:
1. Sampling untreated mucosa
2. Rinsing the mouth with 20 ml of water for 30 seconds
3. Treatment with basic mouthwash (no hyaluronic acid) for 30 seconds
4. Sampling mucosa
5. Rinsing the mouth with 20 ml of water for 30 seconds
6. Treatment with mouthwash containing 0.5% (w/v) of hyaluronic acid according to the present invention for 30 seconds
7. Sampling mucosa
8. Rinsing the mouth with 20 ml of water for 30 seconds
9. Treatment with mouthwash containing 1% (w/v) of hyaluronic acid according to the present invention for 30 seconds
10. Sampling mucosa

Samples were taken by scraping the oral mucosa with a sterile inoculation loop during 15 seconds and scrubbing the loop in 50 μl of water on a glass slide.

The glass slides were allowed to dry for 1 hour at 37° C. in a ventilated oven. The deposit was fixed for 20 minutes with methanol at room temperature. The excess was then removed and the glass slides were dried at room temperature. Hyaluronic acid was stained with Alcian blue for 30 minutes at room temperature and the dye was rinsed several times in water. When blades were dried, pictures were taken by optical microscopy ×20 and analysed qualitatively.

For volunteer 1, an increase of hyaluronic acid staining with the mouthwash containing 0.5% hyaluronic acid compared to the basic mouthwash and to the untreated condition was observed. With 1% hyaluronic acid, there was a strong increase of the stain, proving a deposit of hyaluronic acid on this volunteer's oral mucosa.

For volunteer 2, the staining was lighter, but there was also an increase of the stain correlated with the concentration of hyaluronic acid in mouthwash.

For the volunteer 3, the level of hyaluronic acid on the oral mucosa in the untreated condition was significantly higher than for the other two volunteers. This stain decreased after the water rinsing and treatment with the basic mouthwash, but there was again an increase of the hyaluronic acid staining after treatment with the hyaluronic acid containing mouthwashes.

### Example 11: Evaluation of Toothpaste

Three toothpaste formulations were tested by 33 untrained panellists using sequential monadic product placement with complete block design, fully rotated.

The three toothpaste formulations were blind coded and contained 0% hyaluronic acid, 0.5% hyaluronic acid, and 1.0% hyaluronic acid, respectively. Each panellist tested each toothpaste only once. Brushing was timed to 90 s. After each brushing, the panellists completed a standard closed scale recall questionnaire.

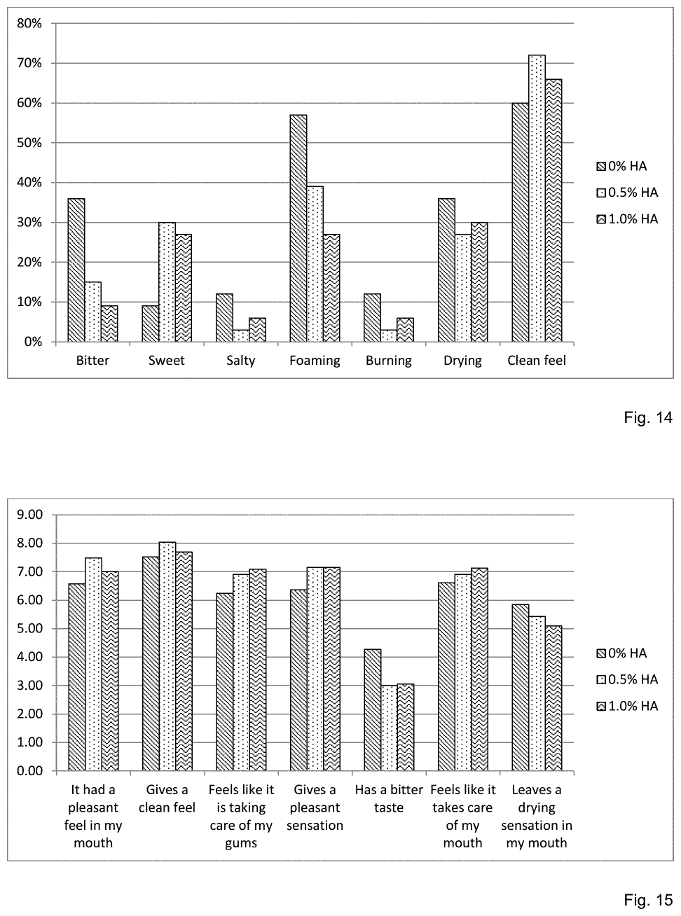

The results are shown in FIGS. 14 and 15.

As can be seen from FIG. 14, the toothpastes containing hyaluronic acid were rated as less bitter, sweeter and less salty than the one without hyaluronic acid. They were also experienced as less foaming, less burning and less drying, and provided a cleaner feel.

FIG. 15 shows more specific perceptions of the different products. In particular, the toothpastes containing hyaluronic acid were found to have a more pleasant feel in the mouth, to give a cleaner feel, to feel more like they were taking care of one's gums, to give a more pleasant sensation, to have a less bitter taste, to feel more like they took care of one's mouth, and to leave a less drying sensation in one's mouth.

1. An oral care composition comprising hyaluronic acid, wherein the hyaluronic acid has an average molecular weight of less than 500 kDa.

2. The oral care composition according to claim 1, wherein the hyaluronic acid has an average molecular weight of 100 to 300 kDa.

3. The oral care composition according to claim 1, wherein the hyaluronic acid has an average molecular weight of 20 to 50 kDa.

4. The oral care composition according to one of claims 1 to 3, claim 1, wherein the hyaluronic acid is provided neat, as a solution or suspension, in encapsulated or mycellized form, adsorbed on particulate surfaces, or is otherwise distributed, In the oral care composition.

5. The oral care composition according to claim 1, which further comprises at least one active ingredient selected from the group consisting of: disinfectants, astringents, haemostatics, oral malodour counteractants, and mixtures thereof.

6. The oral care composition according to claim 1, further comprising at least one thymol glycoside.

7. The oral care composition according to claim 1, wherein the hyaluronic acid is present in a concentration of 0.1 to 1.0% (w/v).

8. An oral care composition effective in promoting oral mucosa repair, comprising hyaluronic acid having an average molecular weight of less than 500 kDa.

9. An oral care composition effective in reducing biofilm formation, comprising hyaluronic acid having an average molecular weight of 100 to 300 kDa.

10. A method of reducing biofilm formation on an oral surface comprising applying an oral care composition according to claim 1 to the oral surface.

11. The oral care composition of claim 6, wherein the at least one thymol glycoside is thymol α-glucoside.

12. The oral care composition of claim 7, wherein the hyaluronic acid is present in a concentration of 0.2 to 0.5% (w/v).

13. A method of increasing the feeling that an oral care composition is taking care of one's gums and/or mouth, comprising the step of:
incorporating hyaluronic acid in the oral care composition, wherein the hyaluronic acid has an average molecular weight of less than 500 kDa.

14. A method of increasing the clean feel of an oral care composition, comprising the step of:
incorporating hyaluronic acid in the oral care composition, wherein the hyaluronic acid has an average molecular weight of less than 500 kDa.

15. A method of reducing the bitter taste of an oral care composition, comprising the step of:
incorporating hyaluronic acid in the oral care composition, wherein the hyaluronic acid has an average molecular weight of less than 500 kDa.

16. A method of reducing the drying sensation of an oral care composition, comprising the step of:
incorporating hyaluronic acid in the oral care composition, wherein the hyaluronic acid has an average molecular weight of less than 500 kDa.

17. A method of improving the pleasant feel of an oral care composition, comprising the step of:
incorporating hyaluronic acid in the oral care composition, wherein the hyaluronic acid has an average molecular weight of less than 500 kDa.
